(19) United States
(12) Patent Application Publication
(10) Pub. No.: US 2011/0123596 A1
(43) Pub. Date: May 26, 2011

(54) SILICA SOL MATERIAL HAVING AT LEAST ONE THERAPEUTICALLY ACTIVE SUBSTANCE FOR PRODUCING BIOLOGICALLY DEGRADABLE AND/OR RESORBABLE SILICA GEL MATERIALS FOR HUMAN MEDICINE AND/OR MEDICAL TECHNOLOGY

(75) Inventors: Iwer Baecker, Duesseldorf (DE); Miranda Rothenburger Glaubitt, Wurzburg (DE); Joern Probst, Kuemach (DE); Holger Egger, Koeln (DE)

(73) Assignee: BAYER INNOVATION GMBH, Duesseldorf (DE)

(21) Appl. No.: 13/054,449

(22) PCT Filed: Jul. 3, 2009
(86) PCT No.: PCT/EP09/04806

§ 371 Date: Feb. 9, 2011

(30) Foreign Application Priority Data
Jul. 16, 2008 (DE) 10 2008 033 327.1

(51) Int. Cl. A61K 9/70; A61K 39/00; C12N 5/071; A61P 37/04; A61P 31/00

(52) U.S. Cl. 424/444; 424/184.1; 435/399; 435/325; 252/182.32

(57) ABSTRACT

The invention relates to a novel silica sol material containing at least one therapeutically active ingredient and its use for the production of bioabsorbable and biodegradable silica gel materials with improved properties. The materials, such as, for example, fibers, fleeces, powder, monolith and/or coating are employed, for example, in medical technology and/or human medicine, in particular for wound treatment.

The invention relates to a novel silica sol material containing at least one therapeutically active ingredient for the production of biodegradable and/or absorbable silica gel materials for human medicine and/or medical technology and to a process for its production and its use. The invention moreover relates to biodegradable and/or bioabsorbable silica gel fiber materials containing at least one therapeutically active ingredient.

There are frequent efforts to develop biodegradable and/or bioabsorbable materials for various applications in human medicine and medical technology. In these areas, continually increasing demands are moreover made, in particular on biocompatibility, biological activity and the toxicological properties of the materials.

Absorbable silica gels are known in the prior art. DE 196 09 551 C1 describes biodegradable, bioabsorbable fiber structures. These fibers can be obtained in a sol-gel process by drawing filaments from a spinning composition and optionally drying these. The spinning composition contains one or more partially or completely hydrolytically condensed compounds of silicon, which are derived by hydrolytic condensation of monomers of the general formula SiX₄. These fibers have the disadvantage that they still show no optimal results in cytotoxicity tests in a degradation directly after the spinning process and must be assessed as cytotoxic in some cases. Such toxicity is generally not desired, especially in use in human medicine or medical technology, for example in the area of wound healing. The process for the production of the fibers according to DE 196 09 551 C1 moreover has the disadvantage that the resulting mixture after the removal of the solvent in the hydrolysis condensation step is already a multiphase mixture and must of necessity be subjected to filtration for the removal of the resulting solid. Moreover, as a result of the formation of the solid phase and as a result of the compulsory filtration step a large amount of the spinnable sol is lost. According to the process of DE 196 09 551 C1, the formation of a not inconsiderable amount of a solid phase, in particular gel formation, cannot definitely be suppressed during maturation either. This again reduces the amount of spinnable sol composition.

Independently of this, it was possible to show that the fibers and fleeces according to the invention have improved wound healing properties. Moreover, the fibers and fleeces according to the invention are particularly suitable for use as cell support structures.

The object of the present invention is to make available a novel silica sol material containing at least one therapeutically active ingredient for the production of biodegradable and/or bioabsorbable silica gel materials. Moreover, the object of the present invention is to make available biodegradable and/or bioabsorbable silica gel materials containing at least one therapeutically active ingredient, which have improved cytotoxicity and/or wound healing properties. A further object can be seen as making available improved cell support structures, for example for in-vitro production of skin implants, cartilage and bone.

The object is achieved by a silica sol material as claimed in claim 1. According to this, a silica sol material containing at least one therapeutically active ingredient can be obtained by
a) carrying out a hydrolysis/condensation reaction of one or more Si compounds of the formula I

SiX₄ (I)
in which the radicals X are identical or different and denote hydroxyl, hydrogen, halogen, amino, alkoxy, acyloxy, alkylcarbonyl and/or alkoxycarbonyl and are derived from alkyl radicals that are optionally substituted straight-chain, branched or cyclic radicals having 1 to 20 carbon atoms, preferably having 1 to 10 carbon atoms, and can be interrupted by oxygen or sulfur atoms or by amino groups, with acidic catalysis at an initial pH of 0 to ≦7, optionally in the presence of a water-soluble solvent, for at least 16 h at a temperature of 0° C. to 80° C.,
b) by subsequent evaporation producing a single-phase solution having a viscosity in the range from 0.5 to 2 Pa·s at a shear rate of 10 s⁻¹ at 4° C.,
c) subsequently cooling this solution and
d) subjecting it to a kinetically controlled maturation, a homogeneous single-phase sol being formed and in one or more of the steps a) to d), preferably in one or more of the steps a) to c) adding at least one therapeutically active ingredient thereto.

In step a), a radical X from one or from two or more different Si compounds of the formula (I):

SiX₄ (I)

is employed in which the radicals X are identical or different and denote hydroxyl, hydrogen, halogen, amino, alkoxy, acyloxy, alkylcarbonyl and/or alkoxycarbonyl and are derived from alkyl radicals that are optionally substituted straight-chain, branched or cyclic radicals having 1 to 20 carbon atoms, preferably having 1 to 10 carbon atoms, and can be interrupted by oxygen or sulfur atoms or by amino groups.

In a preferred embodiment according to the invention, X in the formula (I) represents an optionally substituted straight-chain, branched and/or cyclic alkoxy radical having 1 to 20 carbon atoms, preferably having 1 to 10 carbon atoms. Particularly preferably, X in the formula (I) represents an optionally substituted straight-chain and/or branched C₁-C₅ alkoxy radical. Further particularly preferred radicals are substituted, but preferably unsubstituted straight-chain and/or branched C₂-C₃ alkoxy radicals, such as, for example, ethoxy, N-propoxy and/or isopropoxy.

According to the invention, tetraethoxysilane (TEOS) is very particularly preferably employed as the Si compound in the hydrolysis/condensation reaction according to the invention. The water-soluble solvent employed can preferably be ethanol or a water/ethanol mixture. The Si compound can be employed in a ratio of ≧1 to the ethanol.

The initial pH of 0 to ≦7. preferably of 2 to 5, is adjusted in a preferred embodiment of the invention with and water containing nitric acid. Other acidic mixtures and/or solutions that can generate NO or NO₂ locally, however, are also suitable for carrying out the present invention. These can be, for example, acidic mixtures and/or solutions that with molecular oxygen generate nitrogen monoxide (NO) enzymatically (by means of a nitroxide synthase, NOS) in a physiological environment, which in turn is converted rapidly to NO₂ by the body, or it can also be organic nitrates or nitrate esters (NO donors), e.g. ethyl nitrate, which form NO with the aid of an organic nitrate reductase. For this enzymatic release of NO, thiol groups (cysteine) are needed.

In addition to the dilute nitric acid, according to the invention an aqueous or alcoholic (particularly preferably: an aqueous dilute ethanolic) solution of a physiologically tolerable acid (e.g. citric. succinic, tartaric, acetic or ascorbic acid) and of at least one essential (e.g. L-arginine, particularly preferably; L-valine, L-leucine, L-lsoleucine, L-phenylalanine, L-thyroxine, L-methionine, L-lycine or L-tryptophan) or non-essential amino acid (z.B. L-glutamine, L-glutamic acid, L-asparagine, L-aspartic acid, L-cysteine, L-glycine, L-alanine. L-proline, L-histidine, L-tyrosine) is therefore also suitable as a substrate of the NOS to adjust the pH to the desired value in the weak to medium-strength acidic range.

In a preferred embodiment, the hydrolysis/condensation reaction is carried out with an Si compound and water containing nitric acid in a molar ratio between 1:1.7 to 1:1.9, particularly preferably in a ratio between 1:1.7 and 1:1.8. The water containing nitric acid can be employed as 0.01 N HNO₃.

The hydrolysis/condensation is carried out over a period of at least 16 h, preferably of at least 18 h, at a temperature from 0° C. to 80° C., preferably from 0° C. to 78° C., particularly preferably at 20-60° C., even more preferably at approximately 20° C. to approximately 50° C. and for example—in the case of the use of the materials according to the invention for wound treatment—at room temperature (approximately 20 to approximately 25° C.) or at approximately 37° C.

In a preferred embodiment of the present invention, the hydrolysis can be carried out for a period of at least 16 hours, preferably at least 18 hours to 4 weeks. Preferably, the hydrolysis time is measured at 24 h to 18 days, particularly preferably at 3 to 8 days. It was surprisingly discovered that in the case of a prolonged hydrolysis/condensation time compared to the previously customary times of a few hours at room temperature, after the removal of the solvent in step b) a homogeneous single-phase solution can be obtained, which no longer needs filtration before maturation in step d).

The first hydrolysis/condensation reaction is preferably carried out batchwise in a stirrer vessel or a stop round-bottomed flask with stirrer rod. The Si compound of the formula (I) (e.g. TEOS) and the solvent (e.g. ethanol) are preferably initially introduced. Subsequently, the rapid addition of the acid takes place, preferably in the form of 0.01 N HNO₃ (e.g. 0.01 mol of HNO₃ per mol of TEOS). On account of the acid strength in the reaction mixture, the first hydrolysis/condensation reaction proceeds rapidly, and the contents of the container warm to approximately 40° C., before the temperature begins to fall even during the reaction time (that is in step (a)) (as a result of natural cooling to the surrounding temperature, or heating agent temperature).

The removal of the water-soluble solvent (e.g. ethanol, water) in step (b) is carried out in a preferred embodiment of the invention in a closed apparatus, in which mixing is possible (preferably rotary evaporator and/or stirring vessel) with simultaneous removal of the solvent (water, ethanol) by evaporating at a pressure of 1 to 1013 mbar, preferably at a pressure of <600 mbar, optionally with continuous supply of a chemically inert entraining gas for the partial pressure reduction of the evaporating components of 1-8 m³/h (preferably 2.5 to 4.5 m³/h), a reaction temperature of 30° C. to 90° C., preferably 60-75° C., even more preferably at 60-70° C. and preferably with gentle mixing of the reaction system to 80 rev/min (preferably 20 rev/min to 60 rev/min) up to a viscosity of the mixture of 0.5 to 30 Pa·s at a shear rate of 10 s⁻¹ at 4° C., preferably 0.5 to 2 Pa·s at a shear rate of 10 s⁻¹ at 4° C., particularly preferably about 1 Pa·s (measurement at 4° C., shear rate 10 s⁻¹).

“Entraining gas flow” according to the invention designates a gas flow which is fed to the gas volume via the liquid phase of the reaction system. For the maintenance of the isobaric ratios in the reaction vessel a gaseous volume flow must thereby be led off, which consists both of the “entraining gas” and of the components(s) to be evaporated. The resulting partial pressure decrease, that is the lowering of the amount of the component or of the component mixture to be evaporated in the gas space, increases the driving force for the evaporation of the solvent at the liquid surface.

In a particularly preferred embodiment, the “entraining gas flow” is dispersed by means of a gas disperser suitably arranged in the gas space of the apparatus such that an adequate entraining gas exchange is guaranteed barely above the liquid surface without, however, flowing directly convectively toward the liquid surface. The latter can lead to local gelation in the extreme case, which is undesirable. Gas dispersers by means of which this embodiment can be realized are known to the person skilled in the art.

As a result of the progressive reaction/polymerization (discernible by the viscosity increase) the phase equilibrium shifts, such that the corresponding equilibrium pressure of the solvent in the vapor phase becomes lower and lower. If the equilibrium pressure falls to the overall pressure in the gas phase, the evaporation stops.

In order to evaporate further solvent, the pressure must therefore be optimally lowered, the entraining gas flow adjusted variably and/or the temperature increased.

In a preferred embodiment of the present invention, at least one of the process parameters pressure, entraining gas flow and/or temperature is adjusted variably in terms of time.

In a preferred embodiment of the invention, the evaporation in step b) is carried out at a constant temperature and temporally variable pressure. In a preferred embodiment of the invention, nitrogen and/or air is employed as the chemically inert entraining gas flow for partial pressure reduction.

In a preferred embodiment of the invention, the water-soluble solvent is removed by means of a combination of vacuum and entraining gas flow. The overall pressure and entraining gas flow in this embodiment of the invention can be adjusted independently of one another to be constant or variable in terms of time. In this embodiment of the invention, ideally at least one of the process parameters pressure, entraining gas flow and/or temperature is adjusted to be temporally variable. It is thereby entirely possible, for example, to achieve a certain reaction time with a desired degree of evaporation and/or to adjust the evaporation rate to the reaction kinetics.

In a preferred embodiment of the invention, the evaporation in step b) is carried out at a constant temperature and a temporally variable pressure, where the pressure at the end of the second HCR, starting from normal pressure or slight reduced pressure, is lowered to <600 mbar, preferably <500 mbar, particularly preferably <100 mbar.

In the combination operation mode (vacuum with entraining gas flow), a constant or variable reduced pressure of <600 mbar is preferred.

Temperatures above 60° C. are particularly preferable in order to favor a reductive conversion of the HNO₃ to NO in the otherwise markedly increasing concentration of the HNO₃ in the residual solvent. This very easily volatile gas (normal boiling point approximately −150° C.) is oxidized on contact with air after escaping from the liquid phase to the easily boiling NO₂ (bp approximately 21° C.), which is removed from the system with the waste air or gas flow. In this way, the acid concentration in the material according to the invention is restricted or reduced. Alternatively, the acid strength, however, can also be reduced in one of the following steps, e.g. by aeration of the solid article, e.g. as a fiber fleece.

If, however, the system organic acid/arginine is used in place of nitric acid, the increase in the pH or the decrease in the acid strength is carried out, if desired, for example by means of tris solutions (to the extent that the acid, e.g. acetic acid, cannot be driven out), shortly before application by rinsing in an aqueous tris solution.

Surprisingly, in comparison to DE 196 09 551 C1, it was discovered that by gentle mixing of the reaction system at 20 rev/min to 80 rev/min, the formation of a concentration gradient can be prevented by means of the height of the batch in the reaction vessel during the reactive evaporation (step b). This contributes together with the prolonged hydrolysis/condensation reaction time of at least 16 hours to the fact that in the process according to the invention at least 70%, preferably at least 80% and very particularly preferably at least 90%, of the total reaction batch can be spun.

Step (b) is preferably carried out until a single-phase solution with a viscosity in the range from 0.5 to 2 Pa·s at a shear rate of 10 s⁻¹ at 4° C., preferably about 1 Pa·s (measurement at 4° C., shear rate 10 s⁻¹), is produced.

In a preferred embodiment of the invention, the monitoring of the progress of the reaction in step b) is carried out by means of the viscosity.

The homogeneous and single-phase solution resulting from the hydrolysis/condensation reaction in step b) can subsequently be cooled and advantageously subjected quantitatively and optionally without filtration to a kinetically controlled maturation.

Maturation (step c)) can be carried out according to the invention at a temperature of −20° C. to 10° C., preferably at 2° C. to 4° C. (e.g. in a refrigerator). Particularly preferably, maturation is carried out at 4° C. As a result of the low temperature, a further condensation can proceed during the kinetic control of the maturation time, starting from the Si compounds described above in formula (I). In this mixture, oligomers and/or polymers siloxanes and/or silanols can be formed. The oligomers and/or polymers can also aggregate by means of hydrogen bonds. After maturation, according to the invention an intrinsically viscous homogeneous single-phase sol composition can be formed. Advantageously according to the invention, therefore, the competing formation of a three-dimensional polymer gel network can to the greatest extent be suppressed. A homogeneous sol composition can therefore be obtained that has no solid second phase, in particular to the greatest extent no gel phase.

The maturity time in step d) can be, according to the invention, from 3 days to 4 weeks, preferably at least 10 days, more preferably between 14-40 days, for example between 14 and 28 days, even more preferably at least 25 days and—especially when using the materials according to the invention for wound treatment—between 25 and 40 days. According to the invention, the sol obtained in step d) preferably has a viscosity between 30 and 100 Pa·s (shear rate 10 s⁻¹ at 4° C.) with a loss factor (at 4° C., 10 s⁻¹, 1% deformation) of 2 to 5, preferably of 2.5 to 3.5 (the loss factor is the quotient of viscous to elastic component of the dynamic viscosity). These conditions for maturation are particularly preferable if the silica sol according to step d) is to be spun to give a fiber.

For the production of the silica sol material containing at least one therapeutically active ingredient, in one or more of steps a) to d), preferably in one or more of steps a) to c), at least one therapeutically active ingredient is added thereto.

Therapeutically active ingredients according to the invention preferably used for the production of the silica sol material are in particular selected from the group consisting of the analgesics, anesthetics, antiseptics, hemostyptics, anticoagulatory compounds, antihistaminics, antiphlogistic compounds, plant substances or substance mixtures promoting wound healing, vaccines (e.g. against toxic wound infections), growth factors, regeneration-supporting proteins such as, for example, collagen, enzymes, enzyme inhibitors, in particular protease inhibitors such as, for example, alpha-1-antichymotrypsin and prolastin, vitamins or provitamins, carotenoids, skin-caring compounds, contraceptives and combinations thereof.

Analgesics according to the invention are understood as meaning opioid analgesics, based on prototypes such as morphine, fentanyl and methadone, such as, for example, burprenorphine, and non-opioid analgesics (such as nicotinergic analgesics, such as, for example, epibatidine; acidic antiphlogistic and antipyretic analgesics (NSAID—Non-steroidal anti-inflammatory drugs=non-steroidal anti-inflammatories) such as salicylic acid derivatives, such as, for example, acetylsalicylic acid (ASA), methyl salicylate or diflunisal, phenylacetic acid derivatives such as, for example, diclofenac, 2-phenylpropionic acid derivatives such as, for example, ibuprofen or naproxen; non-acidic analgesics such as 4-aminophenol derivatives such as, for example, paracetamol, pyrazolones such as, for example, metamizole or phenazone, oxicams such as, for example, meloxicam or piroxicam; other non-opioid analgesics such as, for example, flupirtine).

Preferred anesthetics according to the invention are local anesthetics of the amide or ester type, in particular lidocaine, tetracaine, bupivacaine, prilocaine, mepivacaine, etidocaine and also procaine and benzocaine.

Preferred antiseptics according to the invention are selected from the group consisting of the quaternary ammonium compounds such as, for example, benzalkonium cetrimide, cetylpyridinium chloride and octenidine; iodine-containing compounds such as, for example, lodine, lodine-povidone; halogenated compounds such as, for example, triclosan and chlorhexidine; quinoline derivatives such as, for example, oxyquinoline; phenol derivatives such as, for example, resorcinol, triclosan, hexachlorophene; mercury-containing compounds such as merbromine and thiomersal; antimicrobial metals such as, for example, silver, copper or zinc and their salts, oxides or complexes in combination or alone; benzoic acid, benzoyl peroxide and/or biguanides, in particular PHMB.

As an antiseptic, according to the invention those compounds can also be used that have a germicidal, bactericidal (e.g. antibiotics), bacteriostatic (e.g. antibiotics), bacteriolytic (e.g. antibiotics), fungicidal, virucidal, virustatic, anti-parasitic and/or generally microbicidal action.

Hemostyptics preferred according to the invention are selected from the group consisting of thrombin, fibrin, fibrinogen, factor VIII concentrate, vitamin K, PPSB, protamine, antifibrinolytics such as, for example, tranexamic acid and aminocaproic acid.

Anticoagulatory compounds preferred according to the invention are selected from the group consisting of heparin, coumarins, platelet aggregation inhibitors such as, for example, acetylsalicylic acid, cyclooxygenase (COX) inhibitors, clopidogrel, tirofiban; fibrinolytics such as, for example, streptokinase, urokinase, and alteplase.

Antihistaminics according to the invention are selected from the group consisting of the ethylenediamines such as, for example, mepyramine (pyrilamine), tripelennamine (pyribenzamine), antazoline, dimetindene, (bamipine); the ethanolamines such as, for example, diphenhydramine, carbinoxamine, doxylamine, clemastine; the alkylamines such as, for example, pheniramine, chlorphenamine (chlorpheniramine), dexchlorphenamine, brompheniramine, triprolidine; the piperazines such as, for example, hydroxyzine, meclozine; the tricyclic antihistaminics such as, for example, promethazine, alimemazine (trimeprazine), zyproheptadine and azatadine; acrivastine, astemizole, cetirizine, ebastine, fexofenadine, loratadine, mizolastine, terfenadine; azelastine, levocabastine, olopatadine, epinastine, levocetirizine, desloratadine, fexofenadine thioperamide and JNJ7777120.

Antiphlogistic compounds preferred according to the invention are selected from the group consisting of the non-steroidal antiphlogistics/antirheumatics such as, for example, acetylsalicylic acid, diclofenac, diflunisal, flurbiprofen, ibuprofen, indometacin, ketoprofen, mefenamic acid, metamizole, naproxen, oxyphenbutazone, phenylbutazone, phenazone, piroxicam, propyphenazone, salicylamide, tiaprofenic acid, tenoxicam, tolfenamic acid; glucocorticoids such as, for example, clobetasol propionate, triamcinolone acetonide, betamethasone valerate, dexamethasone, prednisolone, prednisone, hydrocortisone, hydrocortisone acetate, fluticasone, budesonide; other antiphlogistics such as, for example, montelukast or plant antiphlogistic extracts from camomile, marigold, arnica.

Plant wound healing-promoting substances or substance mixtures or plant extracts used in connection with the present invention are, in particular, hamamelis extracts e.g. Hamamelis virgina, calendula extract, aloe extract e.g. Aloe vera, Aloe barbadensis, Aloe feroxoder or Aloe vulgaris, green tea extracts, seaweed extract e.g. red algae or green algae extract, avocado extract, myrrh extract e.g. Commophora molmol, bamboo extracts and combinations thereof. Here, according to the invention extracts of the leaves, flowers, stalk or roots of the plants or combinations thereof are particularly to be understood.

Growth factors according to the invention that may be mentioned in particular are: aFGF (acidic Fibroplast Growth Factor), EGF (Epidermal) Growth Factor), PDGF (Platelet Derived Growth Factor), rhPDGF-BB (Becaplermin), PDECGF (Platelet Derived Endothelial Cell Growth Factor), bFGF (basic Fibroplast Growth Factor), TGF α (Transforming Growth Factor alpha), TGF-β (Transforming Growth Factor beta), KGF (Keratinocyte Growth Factor), IGF1/IGF2 (Insulin-Like Growth Factor) VEGF (Vascular Endothelial Growth Factor) and TNF (Tumor Necrosis Factor).

Suitable vitamins or provitamins according to the invention are in particular the fat-soluble or water-soluble vitamins vitamin A, group consisting of the retinoids, provitamin A, group consisting of the carotenoids, in particular β-carotene, vitamin E, group consisting of the tocopherols, in particular α-tocopherol, β-tocopherol, γ-tocopherol, δ-tocopherol and α-tocotrienol, β-tocotrienol, y-tocotrienol and δ-tocotrienol, vitamin K, phylloquinone, in particular phytomenadione or plant vitamin K, vitamin C, L-ascorbic acid, vitamin B 1, thiamine, vitamin B2, riboflavin, vitamin G, vitamin B3, niacin, nicotinic acid and nicotinamide, vitamin B5, pantothenic acid, provitamin B5, panthenol or dexpanthenol, vitamin B6, vitamin B7, vitamin H, biotin, vitamin B9, folic acid and combinations thereof.

Preferred skin-caring compounds according to the invention are in particular antioxidants, light screens, insect repellents, ethereal oils, moisturizers, perfumes and/or coenzyme Q10.

Therapeutically active ingredients according to the invention that can be employed individually or as a mixture of different therapeutically active ingredients are in particular present to 0.01 to 40% by weight, preferably to 0.01 to 20% by weight and very particularly preferably to 0.1 to 10% by weight based on the weight of the silica sol material in the composition.

If the fiber/fleece according to the invention is to be employed for wound healing, the sol obtained in step d) preferably has a viscosity of 35 to 75 Pa·s (shear rate 10 s⁻¹ at 4° C.) and even more preferably of 35 to 45 Pa·s (shear rate 10 s⁻¹ at 4° C.) preferably with a loss factor (at 4° C., 10 s⁻¹, 1% deformation) of 2.5 to 3.5.

Too high a loss factor means too high an elasticity of the material, which, for example, counteracts the formation of a stable filament during spinning (gelation, breaking of the filament). In the case of too low a loss factor the material is so fluid that stable filament formation is not possible (drops).

The conditions during the maturity time can vary provided the silica sol according to the invention is to be processed subsequently to give a powder instead of a spinnable fiber. Preferably, the dynamic viscosity at the end of step (d) is in this case approximately 60 Pa·s (shear rate 10 s⁻¹ at 4° C.).

In the case of processing of the silica sol to give a monolith, the dynamic viscosity at the end of (d) is preferably greater than or equal to 70 Pa·s (shear rate 10 s⁻¹ at 4° C.). If the silica sol is to be used for the coating of bodies or surfaces, the dynamic viscosity, depending on the desired layer thickness, is less than or equal to 10 Pa·s (shear rate 10 s⁻¹ at 4° C.).

Preferably, the sol composition obtained can be employed at least approximately quantitatively in further production steps and/or processes for biodegradable and/or absorbable silica gel materials. Preferably, the sol obtained in step d) is spinnable. In a further step e), a spinning process can be provided according to the invention.

Such a spinning process step can be carried out under customary conditions, such as described, for example, in DE 196 09 551 C1 and DE 10 2004 063 599 A1.

In this process step, that sol is blown out, for example, through a nozzle plate with individual nozzles by a pressure container (pressure in the container 1-100 bar, preferably 20 to 30 bar).

The spinning shaft customarily has a length of 1-5 m advantageously 2 m. The climate in the spinning shaft is adjusted in a controlled manner with respect to temperature and humidity. Temperatures between 20° C. and 30° C. and −5 to 10° C. dew point and from 20 to 40% relative humidity, preferably 20-25% relative humidity and particularly preferably approximately 20% relative humidity, are preferred.

The fibers are dimensionally stable after falling through the spinning shaft and are deposited on a changing bench. The mesh width of the fiber web thus resulting is adjusted, inter alia, by means of the changing speeds. These are a few cm/s. As a result of a two-axis movement a closely meshed fiber web (fleece) thus results, in which, based on TEOS as the Si-containing starting compound, generally additionally over 25 to 33%. of the ethoxy groups are present.

Especially when using the materials according to the invention for wound treatment, the area weight of the fiber material is preferably at least 90 g/m², and particularly preferably at least 150 g/m². The thickness of the wound dressing (consisting of the spun fleece) is preferably at least 0.8 mm and particularly preferably at least 1.5 mm. The fiber diameter is preferably at least approximately 45 μm.

The silica gel fiber materials and products resulting from the process according to the invention, that is, for example, filaments, fibers, fleeces and/or fabric, have outstanding biodegradability and bioabsorption power.

A further advantage according to the invention is that silica gel fiber materials produced according to the invention, compared to fibers that were obtained by the process of DE 196 09 551 C1, have markedly improved values in cytotoxicity tests in tests in the presence of L929 mouse fibroplasts (see Example 1 and comparison example). Products that were produced from the silica sol material according to the invention are therefore distinguished by a particularly good biocompatibility. The filaments, fibers or fleeces according to the invention can inasmuch be employed advantageously as biodegradable and/or bioabsorbable materials and products in human medicine or medical technology.

The fibers and fleeces according to the invention containing at least one therapeutically active ingredient can support or even improve the improved wound healing properties of the fibers and fleeces alone. In particular, the materials according to the invention can therefore be used advantageously in the field of wound treatment and wound healing. Filaments can be employed, for example, as surgical suture material or as reinforcing fibers. Fibrous fleeces according to the invention can be used particularly advantageously in the care of superficial wounds.

The biodegradable and bioabsorbable fibers and fleeces according to the invention can be produced by a controlled hydrolysis/condensation reaction of the abovementioned Si compounds and water acidified with nitric acid by the following steps:
a) a hydrolysis/condensation reaction of one or more Si compounds of the formula I

SiX₄ (I)
in which the radicals X are identical or different and denote hydroxyl, hydrogen, halogen, amino, alkoxy, acyloxy, alkylcarbonyl and/or alkoxycarbonyl and are derived from alkyl radicals that are optionally substituted straight-chain, branched or cyclic radicals having 1 to 20 carbon atoms, preferably having 1 to 10 carbon atoms, and can be interrupted by oxygen or sulfur atoms or by amino groups, is carried out with acidic catalysis at an initial pH of 0 to ≦7, optionally in the presence of a water-soluble solvent, for at least 16 h, preferably for at least 18 h, at a temperature of 0° C. to 80° C., preferably at 20-60° C., particularly preferably at 20 to 50° C., for example at room temperature (approximately 20° C. to approximately 25° C.) or approximately 37° C.,
b) by subsequent evaporation a single-phase solution having a viscosity in the range from 0.5 to 2 Pa·s at a shear rate of 10 s⁻¹ at 4° C. is produced,
c) this solution is subsequently cooled and
d) is subjected to a kinetically controlled maturation, a homogeneous sol being formed and in one or more of the steps a) to d) and preferably in one or more of the steps a) to c) at least one therapeutically active ingredient being added thereto and
e) spinning of the sol obtained in d) in a spinning process.

If, in the hydrolysis/condensation reaction in step a), for example, TEOS is employed as the Si compound, then in the case of adequate hydrolysis time after the evaporation in step b) a homogeneous solution can be obtained. During the maturation time at low temperature, a kinetically controlled reaction can proceed in step c). The mixture can then be present in step d) dissolved as a homogeneous single-phase composition and thus be obtained as a spinnable sol composition.

The fibers or fleeces produced according to the invention can inasmuch be employed advantageously as bioabsorbable and/or bioactive materials in human medicine and/or medical technology. In particular, the materials produced according to the invention can be used advantageously in the field of wound treatment and wound healing. Fibers can be employed, for example, as surgical suture material or as reinforcing fibers. Fleeces can particularly advantageously be used in the care of superficial wounds, in the filtration of body fluids (e.g. blood) or in the field of bioreactors as growth aids.

A further embodiment of the invention can be a drug delivery system and/or a pharmaceutical formulation, a micropowder and/or a nanopowder. It is possible, of course, for further substances adapted to the respective use and/or excipients to be present in the final formulation (powder). The particles of a micropowder according to the invention preferably have a size (a mean diameter) of 0.01 μm to 100 μm, in particular 0.1 μm to 20 μm. The nanopowder particles generally have a size (a mean diameter) of ≦100 nm.

In a further embodiment, the silica sol according to the invention is poured into a mold. After drying, a monolith can be obtained in this way. Such monoliths can be employed in the form of solid implants as an active ingredient supply system (drug delivery system), for example, subcutaneously. They can be employed, for example, as a depot for contraceptives and release the active ingredient over a relatively long period. Such implants according to the invention have good biocompatibility. The monoliths can preferably have a diameter of ≧0.5 mm. Alternatively, the monoliths can also be comminuted and ground to give powders.

According to a further embodiment of the invention, highly viscous sols, in particular hydrogels, can be supplemented or replaced by the silica gel according to the invention. Generally, hydrogels are widely employed in the care of large-area wounds (wound treatment and wound healing). Advantageously, the biocompatibility and thus the wound healing can be improved by the addition of the silica sol. The hydrogels according to the invention can inasmuch be employed advantageously as bioabsorbable and/or bioactive products in medicine, in particular human medicine or medical technology.

The present invention further relates to a process for the in-vitro multiplication of cells, a fiber matrix of a fiber according to the invention serving as a cell support substance and/or guide structure for the extracellular matrix formed by the cells or giving the cells the possibility of finding a spatial arrangement which allows the cells to multiply and/or to achieve their genetically determined differentiation. The advantages of the process according to the invention follow exemplarily from Example 3.

The cells used can be, for example, undifferentiated pluripotent stem cells or cells modified by genetic engineering or native differentiated cells of various differentiation types and degrees.

The cells to be applied to the fiber matrix adhere to the matrix or mainly multiply two-dimensionally on this matrix in order together to form an extracellular matrix or messenger substances (hormones). The fiber matrix preferably forms a surface element, in particular in the form of a fleece or fabric of fibers according to the invention. Preferably, this fiber matrix is porous, such that the introduced/applied cells penetrate it, assume a three-dimensional distribution and according to their genetically determined differentiation or differentiation induced by differentiation factors added thereto can initiate spatial tissue and organ growth or release messenger substances. In an alternative embodiment of the invention, the matrix is formed as a dense fiber network not penetrable by the introduced/applied cells, with the possibility of two-dimensional cell distribution and the simultaneous possibility of three-dimensional tissue and organ growth in the sense of a “composite graft”.

The in-vitro multiplication process according to the invention preferably serves for the in-vitro production of cell conglomerates, tissues and/or organs.

A preferred subject of the invention relates to a cell conglomerate, tissue and/or organs, which can be produced by the process described above. Such a cell conglomerate, tissue and/or organs is/are suitable, for example, as an in vitro model for medicament-tissue-organ interactions. For the production of tissues outside the human body, various processes are used that are summarized under the extensive term “tissue engineering”. For this, depending on tissue type, cells are isolated from their existing tissue conglomerate and multiplied. Afterward, the cells are either applied to flat materials of different consistency or introduced into porous or gelatinous materials, thereby inducing tissue maturation and optionally stimulating by differentiation factors. Tissue maturation can take place outside or within the body. The fiber matrix according to the invention has the advantage here that it is biodegradable and/or bioabsorbable, but—as Example 3 shows—nevertheless almost retains its 2- or 3-dimensional shape for a certain period in the in-vitro multiplication. A preferred subject of the invention accordingly relates to a cell conglomerate, tissue and/or organs having a fiber matrix of polysilicic acid, preferably produced from the fibers according to the invention, the biodegradable and/or bioabsorbable fiber matrix after a period of time of 4 weeks after in vitro cell population for the first time being at least 60%, preferably at least 70% and particularly preferably at least 80%, identical with the original 2- or 3-dimensional shape of the fiber matrix. For example, in the case of such an embodiment the fiber matrix according to the invention degrades and/or absorbs preferably only after application/incorporation of the cell conglomerate, tissue and/or organ to/into an animal or human body.

Depending on cell type, the cells must either be freed beforehand from their matrix conglomerate by enzymatic digestion or by mechanical separation or stimulated to growth by application or introduction to/into a nutrient medium under physiological conditions. The abovementioned fiber matrix functions here as a guide structure for cell growth or as a guide structure for the accumulation of extracellular matrix and tissue constituents. According to the invention, the fiber material can be used in various arrangements. The person skilled in the art knows which arrangement is to be chosen on the basis of the (cell) tissue to be produced. The possible arrangements are the following:
1) as a surface element, i.e. as a dense fiber network that indeed makes possible a penetration extending beyond the range of the applied cells, but still only a limited penetration (i.e. the average size of the holes/fiber or network interstices is in no case larger, preferably even smaller, than the average size of the cells to be cultured; thus the cells can indeed “grow into the fibers”, but only such that they adhere well to the support for the fibers), with the essentially only, but at least principal, possibility of two-dimensional cell distribution and flat cell, tissue and organ growth.
2) as a three-dimensional space element, i.e. as a porous fiber network penetrable by the cells (i.e. the average size of the holes/fiber or network interstices is in no case smaller, preferably even larger, than the average size of the cells to be cultured), with the possibility of three-dimensional cell distribution and spatial cell, tissue and organ growth; 3) as a combination of 1) and 2) in the sense of a “composite graft” or organ by combination of cells, tissues and/or organs and surface sheath tissue (e.g. organ capsule);
3) This variant is possible for tissue structures that are composed of several cell types. For example, vessels consist of endothelium and connective tissue, the endothelium with a flat structure serving for the lining of a blood vessel, while the connective tissue functions as a support substance of the vessel and forms the three-dimensional hollow structure. By means of the combination of 1) as a surface element for the growth of endothelium and 2) as a three-dimensional space element for the growth of connective tissue a vessel can in the end be reconstructed.

Below are listed some tissue and/or cell types that are particularly suitable for multiplication/production by means of one of the three variants and accordingly are preferred according to the invention.

For application 1) preferably the following tissue: epithelium, endothelium, urothelium, mucosa, dura, connective tissue; and preferably the following cells: pluripotent stem cells, chondrocytes (cartilage; for chondrocyte multiplication a two-dimensional medium is needed, for chondrocyte differentiation and cartilage matrix formation, however, a three-dimensional medium is needed. Here, with respect to cartilage only the cells are meant if they dedifferentiate and multiply. Differentiation follows in application 2), osteocytes (bone; either two- or three-dimensional, the same applies here as for the chondrocytes), nerve cells (nerves), hair cells (inner ear hearing organ) or their precursor cells of any differentiation stage (e.g. pluripotent stem cells).

For application 2) the following cells: the cells described for application 1) after their flat multiplication, organ-specific cells (e.g. hepatocytes, nephrocytes, cardiomyocytes, pancreocytes), cells of the CNS with/without endocrine function, e.g. retina, neurocytes, pineal gland, dopaminergic cells, vessel-forming cells (e.g. angiocytes), cells with endo- or exocrine function (e.g. islet cells, adrenal cells, salivary gland cells, epithelial bodies, thyrocytes), cells of the immune system (e.g. macrophages, B cells, T cells or their precursor cells of any differentiation stage such as pluripotent stem cells). The cells of the immune system are cultured three-dimensionally, because in the tissue, after penetration of the blood-tissue barrier they take a three-dimensional structure depending on tissue type) and display their action there in three dimensions.

For application 3) the following lines/tissue/organs: trachea, bronchi, vessels, lymphatic tissue, urethra, ureter, kidney, bladder, adrenal, liver, spleen, heart, vessels, thyroid, tonsils, salivary glands, brain, muscle (smooth, striated), intervertebral discs, meniscus, heart, lung, gall, esophagus, intestine, eye.

A further application possibility of the material used in the invention is the population of the material with cells that have an endo- or exocrine function and release active substances (e.g. hormones, interleukins, inflammatory mediators, enzymes) that display an action in the body or outside the. That is, the material used according to the invention can, on its population with cells with endo- or exocrine function, also serve outside the body for the production of the above-mentioned active substances, which are then made available to the body as medicaments by means of known processes. An action displayed outside the body can serve to influence tissue or cells with the substance released.

A further use of the matrix is that as a bioabsorbable bioimplant as a guide rail for endogenous wound healing under or at the level of the skin, mucosa or in the body interior in the course of operations on organs and tissues. For this, the material, if possible, is incorporated directly or together with further substances into the wound or organ/tissue, for example, by a physician as a surface element or three-dimensional space element during an operation. The properties of the bioabsorbable, inorganic material in the form of fibers used according to the invention require only a small change in the tissue medium for the cells to be cultured, in particular no acidic medium is formed, with the consequence that a negative influence on tissue and organ differentiation is prevented. Furthermore, complete degradation of the material results independently of the pH of the tissue. As a result of the tissue and/or organ reconstruction taking place simultaneously, vital tissue is always found, with the possibility of penetration by anti-infectious medicaments in the case of unwanted population with pathogens (infection). Moreover, the fiber matrix can additionally be treated with further active ingredients of different substance groups with the possibility of a positive influence on tissue and organ differentiation by display of an active and passive action at the site of application, but also by display of action at a remote-lying site of action. For this, the abovementioned therapeutically active ingredients in particular on the one hand contain anti-infectious active ingredients, but on the other hand also active ingredients supporting and modulating the wound healing, the inflammatory reaction and the tissue differentiation, such as, for example, on the one hand growth factors (IGF, TGF, FGF etc.), on the other hand glucocorticoids and interleukins, but also chemotherapeutics and immunosuppressants.

The bioabsorbable, inorganic fibers used according to the invention make possible attachment of the cells used with the possibility of the multiplication of the cells along the fibers, but also with the possibility of the formation of a tissue or organ matrix. At the same time, with the multiplication of the cells or the formation of a tissue or organ matrix, degradation of the fiber structure occurs. Ideally, the tissue, organ or cell construction rate is correlated with the degradation rate of the fiber material by variation of the condensation of the fibers. It holds true here that the less the condensation process (i.e., the elimination of water and thus the polycondensation) has proceeded, all the better the material can be degraded. The highest OH-content and thus the most rapidly degradable fiber is obtained with freshly spun fibers that are subsequently placed into ethanol. The condensation process is moreover influenced by the spinning parameters, i.e. draw-off rate, atmosphere, spinning temperature etc. Fibers produced in this way are biodegradable and bioabsorbable and dissolve in an adjustable period of preferably 2 weeks to 10 weeks, the degradation rate being correlated with the number of silanol groups of the fiber. A further aspect of the present invention relates to the use of the cells, organs and tissue according to the invention, after they have been treated with medicaments and/or active ingredients, as an in vitro model for medicament-tissue-organ interactions. By this means, animal experimental investigations can be minimized or avoided.

A further particularly preferred subject of the invention relates to a process for the production of a skin implant, skins cells being applied to the surface of a nutrient solution and allowed to grow and a surface element made from a fiber according to the invention being laid on the nutrient solution.

The present invention further relates in a preferred subject of the invention to a skin implant consisting of skins cells and a surface element with fibers according to the invention. A surface element (preferably planar) makes possible a flat and thus rapid growth of skins cells, optionally with additional use of further infiltrated medicaments, which are added to the fiber.

The invention will be illustrated in more detail by the following examples, without being restricted thereto.

All viscosities indicated were measured using viscometers from Anton Paar (Physica MCR300 and MCR301 type) at a shear rate of 10 s⁻¹ at 4° C.

### EXAMPLES

### Example 1

### Silica Sol and Bioabsorbable and Biodegradable Silica Gel Material

As starting materials for the hydrolysis/condensation, 4 mol of TEOS (tetraethoxysilane) in ethanol were initially introduced into a reaction vessel and 7 mol of water were added in the form of a 0.01N HNO₃ solution and mixed with one another with stirring. The mixture was stirred at room temperature for 8 days. The solution from the hydrolysis/condensation reaction was subsequently converted during evaporation and condensation in a glass beaker at 70° C. to a nearly water- and ethanol-free solution. This solution was single-phase, contained no solids and had a viscosity of 1 Pa·s (shear rate of 10 s⁻¹ at 4° C.). The solution was cooled to 4° C. and subjected to maturation at this temperature. After a maturation time of 18 days, a homogeneous single-phase sol composition with a viscosity of 43 Pa·s (shear rate 10 s⁻¹ bei 4° C.) was obtained. The sol composition was present without a discernible solid phase content. It was possible to spin the homogeneous sol composition to give fibers. It is also designated as a spinning composition.

The production of the fibers was carried out in a customary spinning unit. For this, the spinning composition was filled into a pressure cylinder cooled to −15° C., which was charged with an air pressure of 20 bar. The force resulting therefrom pressed the sol through nozzles, whereby filaments were formed. Depending on the nozzle diameter, the filaments had a diameter of 5 and 100 μm.

The fluid, honey-like filaments fell as a result of their own weight into a spinning shaft situated under the pressure cylinder and reacted there to give a largely solid form and rigid filaments were formed. The filaments were still reactive on their surface, such that they were able to stick to one another at the contact areas on striking an optionally provided charging bench. As a result of adjustable stroke cycles of the changing bench further crosslinkages resulted between the fibers and a fleece was formed.

Advantageously, the filaments obtained according to the invention were drier than fibers obtained under comparable spinning conditions, which were produced by the process of DE 196 09 551 C1. By this means, in the following production of fleeces, less cross-linked and therefore more flexible fleeces were obtained according to the invention.

The fleece produced according to the invention was subjected to a cytotoxicological test according to ISO 10993-5 (1999); EN 30993-5 (1994). After extraction of the fleece material with DMEM (Dulbecco' s modified Eagle Medium), the extract was sterile filtered and treated with FCS (fetal calf serum; 10% FCS in the extract). This extract treated with FCS was applied under sterile conditions to L929 mouse fibroplast cells and stored for 48 h at 37° C. and a CO₂ partial pressure of 5%.

Triton X 100 was used as a toxic control substance, the cell culture medium as a non-toxic control substance. The cells were fixed for the determination of the cell count and stained with Methylene Blue. After acidic extraction of the Methylene Blue, the dye content was measured by means of photometry and the extinction was compared with a standard curve in order to determine the cell count by means of the dye extinction. The measurement of the cell count in comparison to the control showed that the silica gel material according to the invention had no cytotoxic properties. Measurements of the protein content (after alkaline lysis and protein content determination using the Bradford method) and the release of lactate dehydrogenase (LDH; photometric method) confirmed the results.

### Comparison Example

Under the same conditions, toxicity measurements were carried out with a fleece material that was produced analogously to the example in DE 196 09 551 C1 using a hydrolysis/condensation time of 1.5 h. It was only possible here to spin 50% of the total reaction batch. In the fiber material produced therefrom, cytotoxicity was detected in the cytotoxicity test.

### Example 2

In a further study, five different fibrous fleeces (KG211, KG226, AEH06KGF553, AEH06KGF563 and AEHKGF565 were compared with an absorbable control wound therapeutic (Promogran®) in a wound healing study on guinea pigs running for 3 months.

Differences in the fibrous fleeces result owing to the different production parameters listed in the following Table 1.

TABLE 1

| Parameter/description | | KG211 | | KG226AEH | | 06KGF553 | | AEH06KGF563 | | AEHKGF565 | |
|---|---|---|---|---|---|---|---|---|---|---|---|
| Hydrolysis/condensation | | | | | | | | | | | |
| Apparatus | Type of | 2 1 single neck | | 2 1 single neck | | Stirring | | Stirring | | Stirring | |
| | reaction vessel | round-bottomed | | round-bottomed | | vessel | | vessel | | vessel | |
| | | flask | | flask | | | | | | | |
| | Mixing | Stirring rod | | Stirring rod | | Crossbeam | | Crossbeam | | Crossbeam | |
| Process | Stop criterion/ | 18 h reaction | | 18 h reaction | | 18 h reaction | | 18 h reaction | | 18 h reaction | |
| | aim of the | time | | time | | time | | time | | time | |
| | process step | | | | | | | | | | |
| | Weighing + | 562.49 | g | 562.49 | g | 562.49 | g | 562.49 | g | 562.49 | g |
| | filling | | | | | | | | | | |
| | of TEOS | | | | | | | | | | |
| | Weighing + | 156.8 | g | 156.8 | g | 156.8 | g | 156.8 | g | 156.8 | g |
| | addition of | | | | | | | | | | |
| | ethanol | | | | | | | | | | |
| | Mixing | 15 | min | 15 | min | 15 | min | 15 | min | 15 | min |
| | Weighing + | 60.38 | g | 60.38 | g | 60.38 | g | 60.38 | g | 60.38 | g |
| | preparation | | | | | | | | | | |
| | of water | | | | | | | | | | |
| | Weighing + | 27.81 | g | 27.81 | g | 27.81 | g | 27.81 | g | 27.81 | g |
| | addition | | | | | | | | | | |
| | of IN HNO3 | | | | | | | | | | |
| | Mixing on IN | Swirling | | Swirling | | Swirling | | Swirling | | Swirling | |
| | HNO3 + water | | | | | | | | | | |
| | Temperature | autothermic i.e. after | | autothermic i.e. after | | firstly autothermic, | | firstly autothermic, | | firstly autothermic, | |
| | control | exothermic reaction | | exothermic reaction | | from 3:00 h reaction | | from 0:20 h reaction | | from 0:20 h reaction | |
| | | is conducted at RT) | | is conducted at RT) | | time T = 25° C. | | time T = 70° C. | | time T = 50° C. | |
| Reactive evaporation | | | | | | | | | | | |
| Apparatus | Type of | Rotary evaporator | | Rotary evaporator | | String vessel | | String vessel | | String vessel | |
| | reaction vessel | | | | | | | | | | |
| | Mixing | Rotary evaporator | | Rotary evaporator | | Crossbeam | | Crossbeam | | Crossbeam | |
| | Type of | Water bath | | Water bath | | Double jacket | | Double jacket | | Double jacket | |
| | temperature | | | | | | | | | | |
| | control | | | | | | | | | | |
| | Medium for the | Vacuum | | Vacuum | | Control air | | Control air | | Control air | |
| | entraining flow | | | | | | | | | | |
| | Supply of the | — | | — | | Glass frit | | Glass frit | | Glass frit | |
| | entraining flow | | | | | | | | | | |
| | Removal of | Rotary evaporator | | Rotary evaporator | | Opening in the | | Opening in the | | Opening in the | |
| | the ethanol- | connection | | connection | | lid | | lid | | lid | |
| | containing | | | | | | | | | | |
| | water air | | | | | | | | | | |
| Process | Stop criterion/ | Mass loss | | Mass loss | | dyn. viscosity | | dyn. viscosity | | dyn. viscosity | |
| | aim of the | 61.7% | | 61.7% | | (4° C., 10 s-1): 1 Pas | | (4° C., 10 s-1): 1 Pas | | (4° C., 10 s-1): 1 Pas | |
| | process step | | | | | | | | | | |
| | Mixing | 25 | rev/min | 25 | rev/min | 60 | rev/min | 45 | rev/min | 45 | rev/min |
| | Temperature | 70° | C. | 70° | C. | 60° | C. | 75° | C. | 70° | C. |
| | control | | | | | | | | | | |
| | Airflow | Vacuum to | | Vacuum to | | 3.8 | m3/h | 3.0 | m3/h | 3.0 | m3/h |
| | | about 400 mbar | | about 400 mbar | | | | | | | |
| | Length of | | | | | 05:40 | | 05:30 | | 06:30 | |
| | reactive | | | | | | | | | | |
| | evaporation | | | | | | | | | | |
| | Filtration | Sieve | | Sieve | | Filter | | Filter | | Filter | |
| Maturation | | | | | | | | | | | |
| Apparatus | Maturation | 500 ml PP | | 500 ml PP | | 500 ml PP | | 500 ml PP | | 500 ml PP | |
| | vessel | beaker | | beaker | | beaker | | beaker | | beaker | |
| | Storage during | Refrigerator | | Refrigerator | | Refrigerator | | Refrigerator | | Refrigerator | |
| | maturation | | | | | | | | | | |
| Process | Stop criterion/ | dyn. viscosity | | dyn. viscosity | | dyn. viscosity | | dyn. viscosity | | dyn. viscosity | |
| | aim of the | 39.2, loss | | 41.2; loss | | 45; loss | | 73; loss | | 44; loss | |
| | process step | factor 3.12 | | factor 2.69 | | factor 2.6 | | factor 4.7 | | factor 3.6 | |
| | Maturation | 4° | C. | 4° | C. | 4° | C. | 4° | C. | 4° | C. |
| | temperature | | | | | | | | | | |
| | Type of | static, upright | | static, upright | | static, upright | | static, upright | | static, upright | |
| | storage of the | | | | | | | | | | |
| | maturation | | | | | | | | | | |
| | beaker | | | | | | | | | | |
| | Length of | 28 d | | 39 d | | 11 d | | 10 d | | 19 d | |
| | maturation | | | | | | | | | | |
| (Intermediate) storage | | | | | | | | | | | |
| Apparatus | Storage | 500 ml PP | | 500 ml PP | | 500 ml PP | | 500 ml PP | | 500 ml PP | |
| | vessel | beaker | | beaker | | beaker | | beaker | | beaker | |
| | Site of storage | Deep-freeze | | Deep-freeze | | Deep-freeze | | Deep-freeze | | Deep-freeze | |
| | Storage | −80° | C. | −80° | C. | −80° | C. | −80° | C. | −80° | C. |
| | temperature | | | | | | | | | | |
| | Type of | static, upright | | static, upright | | static, upright | | static, upright | | static, upright | |
| | storage of the | | | | | | | | | | |
| | maturation | | | | | | | | | | |
| | beaker | | | | | | | | | | |
| Spinning | | | | | | | | | | | |
| | Nozzle plate | 7 nozzles, | | 7 nozzles, | | 7 nozzles, | | 7 nozzles, | | 7 nozzles, | |
| | | D = 150 μm | | D = 150 μm | | D = 150 μm | | D = 150 μm | | D = 150 μm | |
| | Spinning tower | about 2 | m | about 2 | m | about 2 | m | about 2 | m | about 2 | m |
| | Changing bench | uniaxial | | uniaxial | | biaxial | | biaxial | | biaxial | |
| | Thawing of a | 01:30:00 | h | 01:45:00 | h | 01:40:00 | h | 01:30:00 | h | 02:00:00 | h |
| | deep-frozen | | | | | | | | | | |
| | sample in the | | | | | | | | | | |
| | refrigerator | | | | | | | | | | |
| | Temperature | | | | | | | | | | |
| | control of | | | | | | | | | | |
| | the spinning | | | | | | | | | | |
| | container | | | | | | | | | | |
| | Waiting period | 03:30:00 | h | 03:00:00 | h | 03:30:00 | h | 02:10:00 | h | 03:00:00 | h |
| | after filling | | | | | | | | | | |
| | the spinning | | | | | | | | | | |
| | container | | | | | | | | | | |
| | Pressure in | 20 | bar | 20 | bar | 30 | bar | 20 | bar | 20 | bar |
| | the spinning | | | | | | | | | | |
| | container | | | | | | | | | | |
| | Temperature | 21° | C. | 22° | C. | 23° | C. | 23° | C. | 22° | C. |
| | in the | | | | | | | | | | |
| | spinning | | | | | | | | | | |
| | tower | | | | | | | | | | |
| | Humidity in | 20% | rh | 33% | rh | 34% | rh | 20% | rh | 22% | rh |
| | the spinning | | | | | | | | | | |
| | tower | | | | | | | | | | |
| | Spinning time | 6 | min | 5 | min | 6 | min | 12 | min | 6 | min |
| | for 1 fleece | | | | | | | | | | |
| | Motion pattern | Stroke length: 28 cm | | Stroke length: 28 cm | | Stroke length: 28 cm | | Stroke length: 28 cm | | Stroke length: 28 cm | |
| | of changing | Stroke cycles: | | Stroke cycles: | | Stroke cycles: | | Stroke cycles: | | Stroke cycles: | |
| | bench | 16/min | | 16/min | | 16/min | | 16/min | | 16/min | |
| Cut | | | | | | | | | | | |
| Apparatus | Cut | 5 × 5 | cm | 5 × 5 | cm | 5 × 5 | cm | 5 × 5 | cm | 5 × 5 | cm |
| | Area Weight | 185 | g/m2 | 165 | g/m2 | about 200 | g/m29 | 0 | g/m2 | 15 | g/m2 |
| Product analysis | | | | | | | | | | | |
| | Thickness of | 1.8 | mm | 2.1 | mm | 1.3 | mm | 0.8 | mm | 1.4 | mm |
| | the wound | | | | | | | | | | |
| | dressing | | | | | | | | | | |
| | Fiber diameter | 44 | μm | 56 | μm | 61 | μm | 45 | μm | 50 | μm |
| | Behavior in | very flexible, | | very flexible, | | Partial fracture, | | No intrinsic | | very flexible, no | |
| | the bending | slight splitting | | slight splitting | | fracture of the | | stability, | | fracture, soft, in | |
| | test | in individual | | in individual | | outermost | | extremely soft | | part splitting in | |
| | | postions | | postions | | positions | | and flexible | | individual positions | |
| | Content of | 0.16 | wt % | 0.79 | wt % | 0.87 | wt % | 0.31 | wt % | 0.67 | wt % |
| | free ethanol | | | | | | | | | | |
| | Ethoxy | 31.3 | wt % | 32.1 | wt % | 27 | wt % | 32.8 | wt % | 33.2 | wt % |
| | group content | | | | | | | | | | |

For the study, dermo-epidermal wounds were surgically prepared in 36 guinea pigs. In each animal, the dermis and epidermis were removed from both sides of the spinal column in an approximate area of 6.25 cm² (2.5×2.5 cm). The wounds were produced by a scalpel. The carneous panniculus was not injured. The wound dressings and Promogran® were laid on the respective wounds. The materials were covered with a nonadhesive wound dressing (URGOTUL®) and a semipermeable adhesive polyurethane film (TEGADERM® or OPSITE®). A cohesive bandage (gauze and ELASTOPLAST®) protected the wound dressings over the wound. Each fibrous fleece or the control material was tested on 5 animals, corresponding to 10 wounds (n=10). At various time intervals, wound healing was evaluated by macroscopic, morphometric and histological investigations.

In the case of all wound dressings tested, no local intolerance was observed. Morphometric investigations showed that those wounds that were treated with Promogran® achieved a 50% wound closure somewhat earlier than those treated with the fleeces. In order to achieve complete (100%) or nearly complete (75%, 95%) wound closure, the time for Promogran® in comparison to most fleeces, however, was somewhat delayed. On average after about 23 days, 100% healing was achieved with KG211 and KG226, for AEH06KGF553, AEH06KGF563 and AEH06KGF565 on average after about 24 days, and for Promogran® only after on average 26 days.

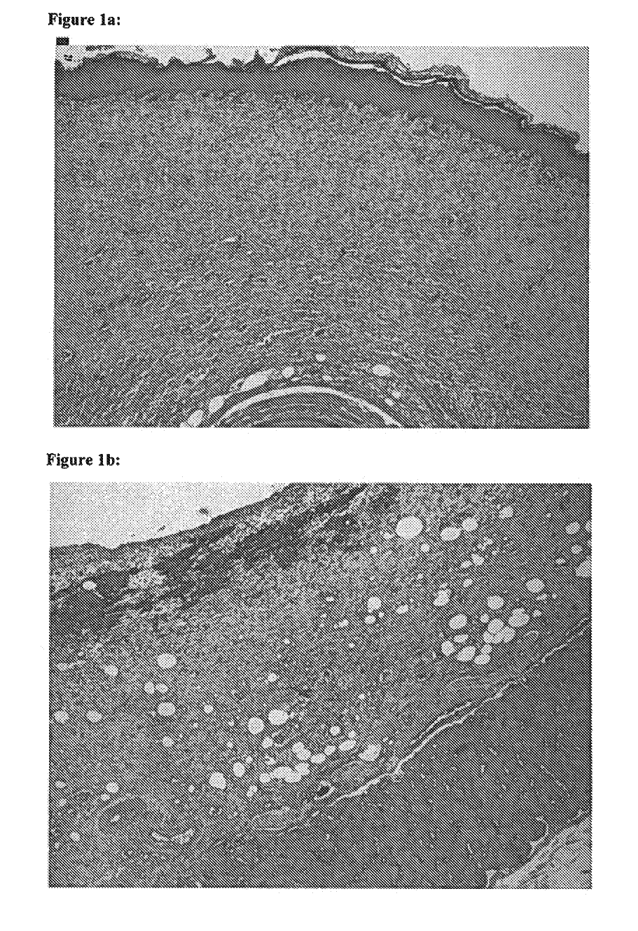

Histological investigations of KG211 animals 28 days after the generation of the wound showed very good wound healing (see FIG. 1a). Only the local tissue reaction was still not completely stabilized, as macrophages were sporadically still to be observed. Independently of this, the granulation tissue was unremarkable, showed a normal thickness and was covered by a newly formed closed epithelial layer.

Histological investigations of the Promogran® animals 28 days after generation of the wound showed granulation tissue that was strongly vacuolized and permeated by polymorphonuclear cells (see FIG. 1b). In contrast to KG211, the granulation tissue was not covered by an epithelial layer.

The wound dressings accordingly show shortened wound healing with simultaneous generation of a better granulation layer and a minimization of inflammatory processes in comparison to Promogran® in the first 4 weeks of wound healing.

### Example 3

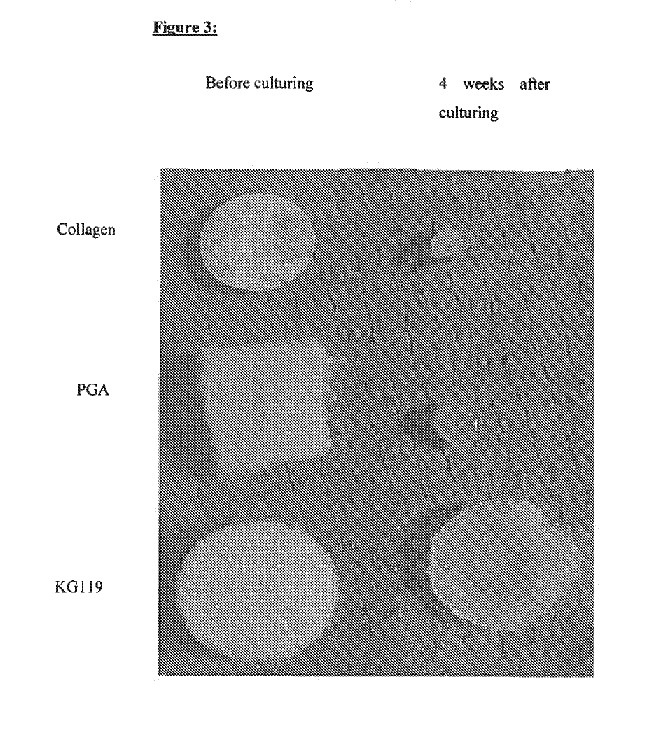

The fiber matrix KG119 of biodegradable and/or bioabsorbable fibers as a cell support substance and also collagen and polyglycolic acid (PGA) were sterilized with gamma rays and placed in an incubator for one hour in a complete medium. The fiber matrix KG119 concerns as a surface element a fleece. It was produced according to the process parameters shown in Table 2. The cut was stamped out in a circular manner (see FIG. 3):

TABLE 2

| Parameter/description | | KG119 | |
|---|---|---|---|
| Hydrolysis/condensation | | | |
| Apparatus | Type of reaction vessel | 2 1 single neck round- | |
| | | bottomed flask | |
| | Mixing | stirring rod | |
| Process | Stop criterion/aim of the process step | 18 h reaction time | |
| | Weighing + filling of TEOS | 562.49 | g |
| | Weighing + addition of ethanol | 156.8 | g |
| | Mixing | 15 | min |
| | Weighing + preparation of water | 60.38 | g |
| | Weighing + addition of 1N HNO3 | 27.81 | g |
| | Mixing of 1N HNO3 + water | swirling | |
| | Temperature control | autothermic | |
| Reactive evaporation | | | |
| Apparatus | Type of reaction vessel | open PP beaker | |
| | Mixing | none | |
| | Type of temperature control | water bath | |
| | Medium for the entraining flow | compressed air | |
| | Supply of the entraining flow | transverse flow | |
| | | through beaker | |
| | Removal of the ethanol-containing | uncontrolled to | |
| | waste air | the environment | |
| Process | Stop criterion/aim of the process step | mass loss | |
| | | 61.7% | |
| | Mixing | 0 | |
| | Temperature control | 70° | C. |
| | Airflow | uncontrolled to | |
| | | the environment | |
| | Reactive evaporation time | mass loss | |
| | Filtration | sieve | |
| Maturation | | | |
| Apparatus | Maturation vessel | 500 ml PP beaker | |
| | Storage during maturation | refrigerator | |
| | Determination of maturation progress | | |
| | In-process control | sieve | |
| Process | Stop criterion/aim of the process step | dyn. viscosity, 30 | |
| | | Pas before spinning, | |
| | | loss factor 3.22 | |
| | Maturation temperature | 4° | C. |
| | Type of storage of the maturation | static, upright | |
| | beaker | | |
| (Intermediate) storage | | | |
| Apparatus | Storage vessel | 500 ml PP beaker | |
| | Site of storage | deep-freeze | |
| | Storage temperature | −80° | C. |
| | Type of storage of the maturation | static, upright | |
| | beaker | | |
| Spinning | | | |
| | Nozzle plate | 7 nozzles, D = 150 μm | |
| | Set-up for rinsing the nozzle plates | after 1 h | |
| | Spinning tower | about 2 m | |
| | Changing bench | uniaxial | |
| | Temperature in the spinning tower | RT | |
| | Humidity in the spinning tower | about 30% rh | |
| | Spinning time for 1 fleece | 6 | min |
| | Movement pattern of changing bench | stroke length: 28 cm | |
| | | stroke cycles: | |
| | Conditioning of the fleece | 6 | min |

| | |
|---|---|
| Cut | 2.5 × 2.5 cm |

Before cell population the medium was renewed. Human dermal fibroblast cells were then added. Cell culture was carried out in 24-hole Falcon 351147 plastic plates. The medium was changed every day. The cell population medium was Gibco Dulbecco's Modified Eagle's Medium 42430-250 supplemented with 10% fetal calf serum (FCS) and 100 units/ml of penicillin, 0.25 μg/ml of amphotericin B and 0.1 mg/ml of streptomycin as antibiotics. During the growth of the cells, after the initial medium change 50 μg/ml of ascorbic acid were added to the medium. It was further necessary in view of the increasing cell count to treat the medium with a sodium bicarbonate solution buffer (7.5% Sigma). The cell standards (control cells without cell support substance) were cultured in customary tissue culture dishes and glass bottom Iwaki plates.

The Alamar Blue assay was carried out with reagents from Serotec. These were diluted to 10% with HBSS (phenol-free) buffer, adjusted to 37° C. and sterile filtered. The cell support substances with the cells were washed in PBS and then removed from their original plates and placed in tissue culture dishes and glass bottom Iwaki plates.

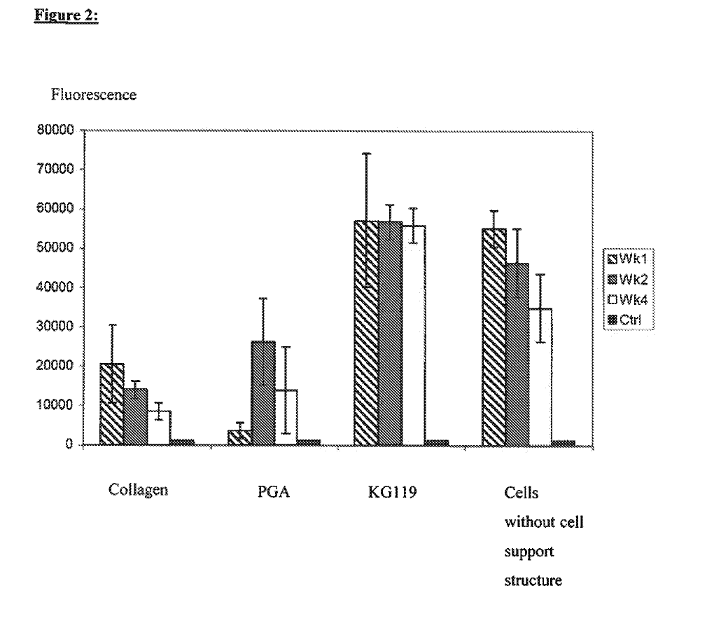

The metabolic activity measured using the Alamar Blue assay is a function of the cell count and the metabolic activity of the individual cells. FIG. 2 compares the activity (shown in the form of a measured fluorescence value) of the dermal fibroblasts on the various matrices collagen, PGA and the fiber matrix KG119, and cells without support structure (control culture, Ctrl) in the case of a culture period of one week (Wk 1), 2 weeks (Wk 2) and 4 weeks (Wk 4).

The primary adhesion of the cells to KG119 is strong and comparable to that of collagen. KG119 and collagen excel PGA with regard to cell adhesion (data not shown). The longer the cells grow on the matrices, the more clearly the superiority of the fiber matrix KG119 is seen. FIG. 2 shows that KG119 excels the other cell support structures with regard to metabolic activity of the cells. The high metabolic activity is retained over the whole measurement period (4 weeks). In contrast to this, collagen, PGA and cells without cell support structures cannot maintain the metabolic activity over this period of time. Only KG119 shows high cell adhesion and cell proliferation with retention of the metabolic activity over the whole period.

FIG. 3 shows the cell support structures collagen, PGA and KG119 before culturing with human dermal fibroblast cells and after a culture period of 4 weeks. Collagen and PGA cell support structures contract and degrade to give a dense tissue ball. Only KG119 retains its original shape. Inside KG119, a dense dermal tissue mass has formed and the fibers are firmly connected to the tissue.

### Example 4

### Production of Silica Sol Containing Therapeutically Active Ingredient

In a temperature-controllable reaction vessel, 5.4 mol of tetraethoxysilane were initially introduced into ethanol. The solution was homogenized for about 15 min. Subsequently, 56 g of 1 N nitric acid diluted with 121 g of water are added with stirring. The mixture was stirred for 18 h. The reaction here initially proceeded autothermically and was temperature-controlled at 37° C. for 2 h after addition of acid.

Subsequently, 12 g of lidocaine were added with moderate stirring of the mixture. This mixture was transferred to a preheated temperature-controllable reaction vessel and evaporated to about 38.5% of its original mass over a period of 6 h at about 64° C. and with slight stirring. The solution was single-phase, contained no solids and had a viscosity of about 3 Pa s (shear rate of 10 s⁻¹ at 4° C.).

The solution was cooled to 4° C. and subjected to maturation at this temperature.

### Example 5

In a 2 l flask 1124.98 g of TEOS are mixed with 313.60 of EtOH with stirring for 15 min.

In a 250 ml glass beaker, 120.76 g of H2 O, 55.62 g of 1N HNO3 and 0.12 g of a nanosilver solution (AgPURE™ W 5%, rent a scientist GmbH) are intensively stirred and optionally dispersed for 15 min in ultrasound and added to the diluted TEOS. This batch is stirred for a total of 18 h as standard (2 h at room temperature, subsequently 16 h in a warm water bath at 40° C.). After the 18-hours reaction period, a few black particles are to be seen in the sol, after a 30-minute ultrasound treatment a sol is obtained having only very slight turbidity. This sol is reactively evaporated down to a weight loss of 61.7% in the reactor at 70° C. At 4° C., the sol matures to the spinning composition until the desired rheological data are achieved. The sol can be spun well, and the wound dressings have a very slight gray coloration in comparison to the undoped wound dressing.

1. A silica sol material comprising at least one therapeutically active ingredient obtainable by
a) conducting a hydrolysis/condensation reaction of one or more Si compounds of the formula (I)
SiX₄ (I)
in which the radicals X are identical or different and denote hydroxyl, hydrogen, halogen, amino, alkoxy, acyloxy, alkylcarbonyl and/or alkoxycarbonyl and are derived from alkyl radicals that are optionally substituted straight-chain, branched or cyclic radicals having 1 to 20 carbon atoms, and can be interrupted by oxygen or sulfur atoms or by amino groups,
with acidic catalysis at an initial pH of 0 to ≦7, optionally in the presence of a water-soluble solvent, for at least 16 h at a temperature of 0° C. to 80° C.,
b) subsequent evaporation a single-phase solution having a viscosity in the range from 0.5 to 2 Pa·s at a shear rate of 10 s⁻¹ at 4° C. is produced,
c) this solution is subsequently cooled and
d) is subjected to a kinetically controlled maturation, a homogeneous sol being formed and in one or more of the steps a) to d) and said at least one therapeutically active ingredient being added thereto.

2. The silica sol material as claimed in claim 1, wherein for the acidic catalysis H₂O acidified with nitric acid is employed in a molar ratio in the range 1:1.7 to 1:1.9, and the hydrolysis/condensation reaction is carried out for at least 16 h, and between 20 and 60° C.

3. The material as claimed in claim 1, wherein the hydrolysis/condensation reaction in step a) is carried out at 20 to 60° C., over a period of at least 16 h to 4 weeks.

4. The material as claimed in claim 1, wherein the step b) proceeds in a closed apparatus at a reaction temperature of approximately 30 to approximately 90° C.

5. The material as claimed in claim 1, wherein the solution in step c) is cooled to 2° C. to 4° C.

6. The material as claimed in claim 1, wherein the maturation in step d) takes place at a temperature from 2° C. to 4° C.

7. The material as claimed in claim 1, wherein in step d), the maturation is carried out up to a viscosity of the sol of 30 to 100 Pa·s at a shear rate of 10 s⁻¹ at 4° C. and a loss factor of 2 to 5 (at 4° C., 10 s⁻¹, 1% deformation).

8. The material as claimed in claim 1, wherein the Si compound employed in step a) is tetraethoxysilane.

9. A material as claimed in claim 1 as a material for the production of a biodegradable and/or bioabsorbable silica gel material.

10. A material as claimed in claim 1 as a spinning material for the production of a biodegradable and/or bioabsorbable fiber and fleece in human medicine and/or medical technology optionally for wound treatment and/or wound healing.

11. A material as claimed in claim 1 as a material for the production of a bioabsorbable and/or bioactive powder, monolith and/or coating.

12. A bioabsorbable and/or bioactive powder, monolith and/or coating, which is prepared by at least one further step starting from the silica sol material as claimed in claim 1.

13. A biodegradable and/or bioabsorbable fiber material, wherein a silica sol as claimed in claim 1 is subsequently spun in a spinning process.

14. The biodegradable and/or bioabsorbable fiber material as claimed in claim 13, wherein the fiber material comprises fibers, endless filaments, fleeces and/or fabric.

15. A process for the production of a silica sol material spinnable to at least 70% of the total reaction batch, by
a) an at least 16-hour hydrolysis/condensation reaction of one or more
Si compounds of the formula (I)
SiX4 (I)
in which the radicals X are identical or different and denote hydroxyl, hydrogen, halogen, amino, alkoxy, acyloxy, alkylcarbonyl and/or alkoxycarbonyl and are derived from alkyl radicals that are optionally substituted straight-chain, branched or cyclic radicals having 1 to 20 carbon atoms, and can be interrupted by oxygen or sulfur atoms or by amino groups,
b) evaporation for the production of a single-phase solution optionally with a simultaneous gentle mixing of the reaction system,
c) cooling of the single-phase solution and
d) kinetically controlled maturation for the production of the silica sol material.

16. A process for the in-vitro multiplication of cells, comprising using a fiber matrix of fibers as claimed in claim 13 serving as a cell support substance and/or guide structure for an extracellular matrix formed by the cells.

17. A cell conglomerate, tissue and/or organs, which can be produced according to the process as claimed in claim 16.

18. A cell conglomerate, tissue and/or organs having a fiber matrix of polysilicic acid, the biodegradable and/or bioabsorbable fiber matrix after a period of time of 4 weeks after in-vitro cell population for the first time being at least 60% identical with the original 2- or 3-dimensional shape of the fiber matrix.
